(19) United States
(12) Patent Application Publication
(10) Pub. No.: US 2014/0257199 A1
(43) Pub. Date: Sep. 11, 2014

(54) MEDICAL APPARATUS FACILITATING VASCULAR ANALYSIS AND TREATMENT

(71) Applicant: THE GENERAL HOSPITAL CORPORATION, BOSTON, MA (US)

(72) Inventor: Steven Wu, Lexington, MA (US)

(21) Appl. No.: 14/351,251

(22) PCT Filed: Oct. 15, 2012
(86) PCT No.: PCT/US2012/060191

§ 371 Date: Apr. 11, 2014

Related U.S. Application Data
(60) Provisional application No. 61/547,477, filed on Oct. 14, 2011.

(51) Int. Cl. A61M 1/36

(52) U.S. Cl. CPC A61M 1/3661
USPC 604/256

(57) ABSTRACT

A medical apparatus generally includes a housing that translatably supports a grasping member and a motion control mechanism that is disengagable to permit motion of the grasping member relative to the housing. The housing is configured to support a flow inhibiting instrument when the grasping member is disposed in a retracted position. As such, the medical apparatus facilitates effective blockage of venous outflow from a hemodialysis access structure. In addition, the grasping member translates to a receiving position in which various vascular instruments may be inserted into the grasping member and to a holding position in which the grasping member firmly grasps the instrument. By holding the end of the apparatus opposite the instrument, a practitioner may position his or her hands outside of a medical imaging field and thereby reduce radiation exposure to the hands.

This application claims the benefit of U.S. Provisional Patent Application No. 61/547,477 filed Oct. 14, 2011, the disclosure of which is hereby incorporated by reference.

### STATEMENT OF FEDERALLY SPONSORED RESEARCH OR DEVELOPMENT

Not applicable.

### BACKGROUND OF THE DISCLOSURE

The present disclosure generally relates to a medical apparatus for supporting various types of vascular instruments and, more particularly, to a medical apparatus for supporting instruments used to analyze and treat hemodialysis access structures.

Hemodialysis is a procedure for removing waste material from a subject's body, for example, during renal failure. Such a procedure requires a bodily access structure through which blood is removed from the subject, delivered to a hemodialysis machine for waste removal, and then returned to the subject. Preferred access structures typically facilitate rapid blood flow to the hemodialysis machine and are created surgically. One exemplary structure is an arteriovenous fistula, which is a direct connection between a subject's vein and artery, for example, the cephalic vein and the radial artery in the forearm, created via anastomosis. Another exemplary structure is an arteriovenous graft, which is similar to a fistula, although an artificial vessel joins the subject's vein and artery.

Regardless of the specific type, hemodialysis access structures are frequently tested for clots, stenoses, or other blockages that could reduce the blood flow rate therethrough. To investigate arterial inflow to the structure, a surgeon, radiologist, or other clinician (generally referred to herein as a “practitioner”) typically blocks venous outflow using one or more devices, such as intravascular balloons, tourniquets, and/or blood pressure cuffs.

Unfortunately, the above devices each have various drawbacks. For example, an intravascular balloon may be advantageously used if the access structure has a stenosis that requires angioplasty. Conversely, however, use of an intravascular balloon is difficult to justify if another type of surgical procedure, such as angioplasty, is not required. As another example, both tourniquets and blood pressure cuffs are relatively ineffective for blocking venous outflow due to, in some cases, the presence of collateral veins. Tourniquets can also cause subject discomfort, and blood pressure cuffs can only be placed in certain locations due to their size and lack of sterility.

Furthermore, analysis and treatment of hemodialysis access structures sometimes includes medical imaging procedures so that clots, stenoses, or other blockages can be visualized. The practitioner may simultaneously employ an elongated wire, an access sheath, or other instruments to interact with and treat these blockages. However, such simultaneous actions may expose the practitioner's hands to radiation.

Considering the above, what is needed in the art is a medical apparatus that addresses one or more of the drawbacks of devices used to analyze and treat hemodialysis access structures.

### SUMMARY OF THE INVENTION

In one aspect, the present invention provides a medical apparatus for supporting various medical instruments. These medical instruments are particularly useful for analysis and treatment of hemodialysis access structures, although they may also be used in other manners.

In another aspect, the present invention provides a medical apparatus for supporting a vascular instrument configured to interact with a portion of a cardiovascular system of a subject. The apparatus comprises a housing that defines an internal chamber, and the internal chamber has an opening defined at an end of the housing. A grasping member is supported by the housing within the internal chamber, and the grasping member is translatable relative to the housing to at least partially protrude from the internal chamber through the opening and support the vascular instrument. A motion control mechanism is supported by the housing and is engagable to inhibit translation of the grasping member relative to the housing.

In another aspect, the present invention provides a medical apparatus for supporting a first vascular instrument configured to interact with a portion of a cardiovascular system of a subject and for supporting a second vascular instrument configured to inhibit blood flow through the portion of the cardiovascular system. The apparatus comprises a housing that defines an internal chamber, and the internal chamber has an opening defined at an end of the housing and is configured to detachably receive the second vascular instrument. A grasping member is supported by the housing within the internal chamber and is movable from a retracted position to a receiving position relative to the housing. A motion control mechanism is supported by the housing and is engagable to inhibit translation of the grasping member relative to the housing. In the receiving position at least a portion of the grasping member protrudes from the internal chamber through the opening and is configured to support the first vascular instrument.

In yet another aspect, the present invention provides a medical apparatus configured to interact with a portion of a cardiovascular system of a subject. The apparatus includes an elongated housing and a flow inhibiting instrument detachably supported by the housing and being configured to engage the portion of the cardiovascular system and inhibit blood flow therethrough.

The foregoing and other objects and advantages of the invention will appear in the detailed description that follows. In the description, reference is made to the accompanying drawings that illustrate a preferred configuration of the invention.

### BRIEF DESCRIPTION OF THE DRAWINGS

FIG. 1 is a side view of a medical apparatus according to the present invention;

FIG. 2 is a top view of the medical apparatus of FIG. 1;

FIG. 3 is a partial section view of the medical apparatus along line 3-3 of FIG. 2;

FIG. 4 is a section view of a housing of the medical apparatus along line 3-3 of FIG. 2;

FIG. 5 is a side view of a grasping member of the medical apparatus separated from the housing;

FIG. 6 is a partial section view of the medical apparatus with the grasping member moving away from the retracted position of FIG. 1;

FIG. 7 is a partial section view of the medical apparatus with the grasping member in an instrument receiving position;

FIG. 8 is a partial section view of the medical apparatus with the grasping member moving away from the receiving position of FIG. 7;

FIG. 9 is a partial section view of the medical apparatus with the grasping member in an instrument holding position;

FIG. 10 is a view of the medical apparatus guiding an instrument toward the inflow of an arteriovenous fistula of a subject;

FIG. 11 is a partial section view of the medical apparatus with a flow inhibiting instrument connected to the housing;

FIG. 12 is an end view of the flow inhibiting instrument of FIG. 11;

FIG. 13 is an end view of a second embodiment of the flow inhibiting instrument of FIG. 11;

FIG. 14 is an end view of a third embodiment of the flow inhibiting instrument of FIG. 11;

FIG. 15 is an end view of a fourth embodiment of the flow inhibiting instrument of FIG. 11;

FIG. 16 is a view of the medical apparatus inhibiting blood flow through the vein of an arteriovenous fistula of a subject;

FIG. 17 is a view of the medical apparatus guiding a wire from the vein of an arteriovenous fistula of a subject to the artery of the fistula;

FIG. 18 is a partial section view of an alternative configuration of a medical apparatus according to the present invention;

FIG. 19 is a side view of an alternative embodiment of the housing of the medical apparatus according to the present invention;

FIG. 20 is a top view of the housing of FIG. 19;

FIG. 21 is a partial section view of another alternative embodiment of the medical apparatus according to the present invention in the receiving position;

FIG. 22 is a partial section view of the embodiment of the medical apparatus of FIG. 21 in the holding position; and

FIG. 23 is a partial section view of another alternative embodiment of the medical apparatus according to the present invention.

### DETAILED DESCRIPTION OF THE INVENTION

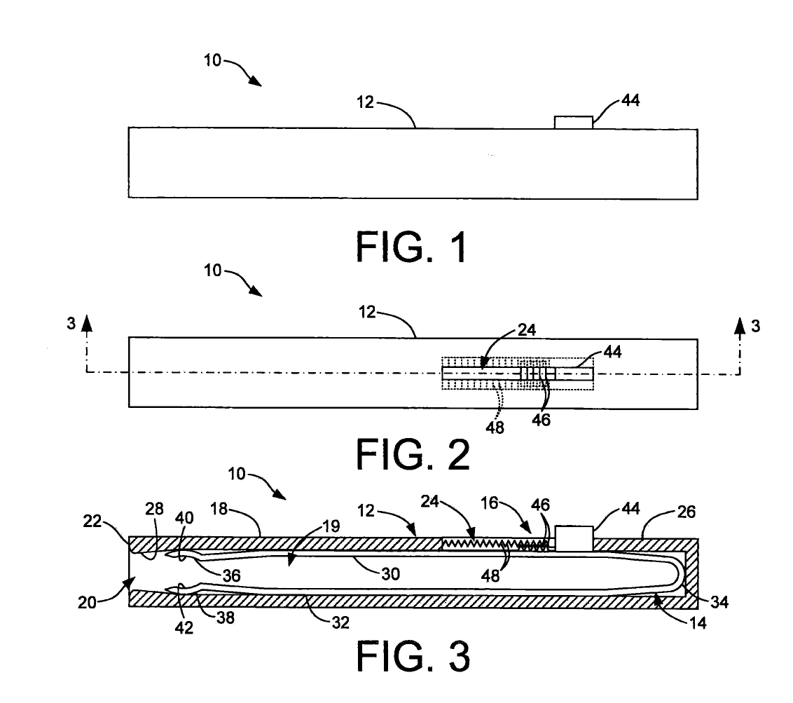

Referring first to FIGS. 1-3, 7, and 9, a medical apparatus 10 according to the present invention generally includes a housing 12 that translatably supports a grasping member 14 and a motion control mechanism 16 (FIG. 3) that is disengagable to permit motion of the grasping member 14 relative to the housing 12. The housing 12 and the grasping member 14 are both capable of interacting with vascular instruments used, for example, during hemodialysis access structure-related medical procedures. For example, the housing 12 is configured to support flow inhibiting instruments when the grasping member 14 is disposed in a retracted position (that is, disposed completely within the housing 12 as shown in FIGS. 1-3). As such, the medical apparatus 10 facilitates effective blockage of venous outflow from a hemodialysis access structure. In addition, the grasping member 12 translates to a receiving position (FIG. 7) in which various vascular instruments (for example, wires and access sheaths) may be inserted into the grasping member 12 and to a holding position (FIGS. 9) in which the grasping member 12 firmly grasps the instrument. By holding the end of the apparatus 10 opposite the instrument, a practitioner may position his or her hands outside of a medical imaging field and thereby reduce radiation exposure to the hands.

In the following paragraphs, the structures of the housing 12, the grasping member 14, and the motion control mechanism 16 are first described in further detail. The interactions of the housing 12 and the grasping member 14 with the vascular instruments are then described, and several exemplary alternative configurations of the medical apparatus 10 are described thereafter.

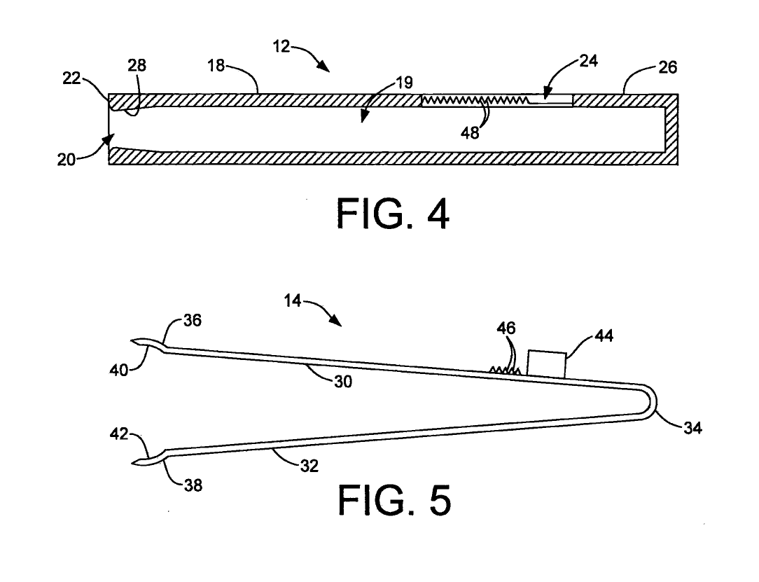

Referring now to FIGS. 3 and 4, the housing 12 is generally an elongated sleeve-like or tube-like component that has an overall length in the range of, for example, 5 cm to 75 cm and an overall diameter in the range of, for example, 0.4 cm to 10 cm. The housing 12 comprises any of various appropriate materials, such as medical grade plastics or the like.

The housing 12 includes a wall 18 that defines an elongated internal chamber 19 in which the grasping member 14 is disposed. The wall 18 defines a first opening 20 between the internal chamber 19 and the outside of the housing 12 on an end 22 of the housing 12 that faces in the longitudinal direction of the apparatus (that is, a direction along the length or elongated dimension of the housing 12). The grasping member 14 protrudes through the first opening 20 in the receiving and holding positions. The wall 18 also defines a second opening 24 on a side 26 of the housing facing a transverse direction (that is, a direction extending perpendicularly to the longitudinal direction). This opening 24 permits the practitioner to access and move the grasping member 14 when the grasping member 14 is disposed in the retracted, receiving, or holding positions.

The internal chamber 19 generally has a constant cross-sectional shape over the majority of its length. However, the wall 18 also defines a narrowed or tapered section 28 that reduces the cross-sectional area of the internal chamber 19 proximate the opening 20. This tapered shape permits the housing 12 to engage the flow inhibiting instrument and deflect portions of the grasping member 14 as described in further detail below.

Turning now to FIGS. 3 and 5, the grasping member 14 is generally an elongated component that comprises any of various flexible materials, such as elastically deformable plastics, elastically deformable metals, or the like. Prior to insertion into the housing 12 (FIG. 5), the grasping member 14 defines a general V-shape in which an upper arm 30 and a lower arm 32 are disposed further apart from each other proceeding away from a curved back portion 34 connecting the arms 30, 32. The arms 30, 32 are forced toward each other when the grasping member 14 is positioned in the housing 12; as such, the arms 30, 32 are preloaded and thereby tend to be spaced apart when the grasping member 14 is positioned in the housing 12.

Opposite the back portion 34 and proximate the first opening 20 of the housing 12, each arm 30, 32 terminates at a finger 36, 38. Due to the unloaded shape of the grasping member 14 as described above, the fingers 36, 38 are biased apart from each other such that various vascular instruments may be inserted therebetween in the receiving position (FIG. 7). In addition, the fingers 36, 38 have arcuate shapes in which the concave surfaces 40, 42 face each other such that fingers 36, 38 may enclose or firmly “grasp” vascular instruments of various sizes in the holding position (FIG. 9).

The grasping member 14 also includes a manipulation button or tab 44 that facilitates practitioner interaction with the grasping member 14. The manipulation tab 44 is supported by the upper arm 30 and is a generally three dimensional rectangular shape that extends through the second opening 24 to the outside of the housing 12. As such, the practitioner may move the manipulation tab 44 to move the grasping member 14 from the retracted positions to the receiving and holding positions. Furthermore, the practitioner may press the manipulation tab 44 downwardly when the grasping member 14 is in the receiving position to grasp an instrument between the fingers 36, 38.

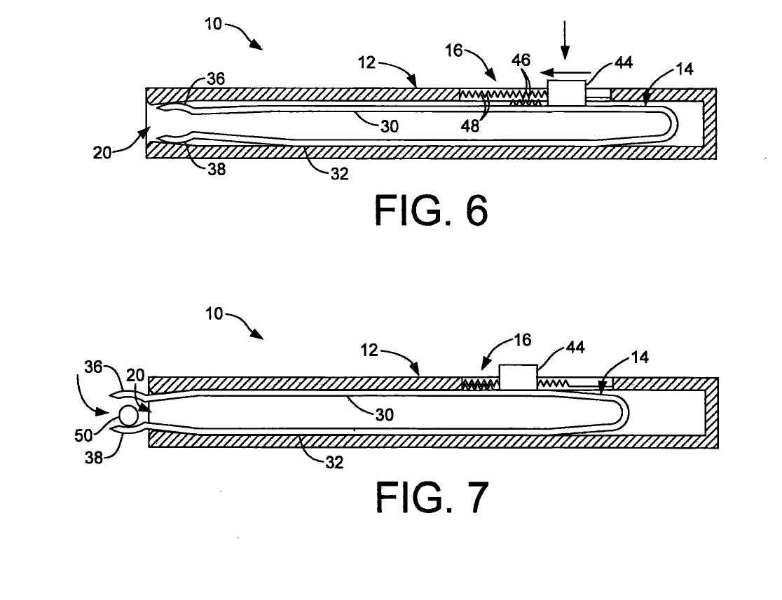

Referring to FIGS. 3-5 and as briefly described above, the motion control mechanism 16 normally inhibits motion of the grasping member 14 in the longitudinal direction within the internal chamber 19. To this end, the motion control mechanism 16 includes a plurality of transversely upwardly facing teeth 46 supported by the upper arm 30 of the grasping member 14 and facing away from the lower arm 32. The teeth 46 interdigitate with a plurality of transversely downwardly facing teeth 48 defined by the housing wall 18 to the sides of the second opening 24. The teeth 46, 48 normally engage each other because the preloaded grasping member 14 holds the upwardly facing teeth 46 in engagement with the downwardly facing teeth 48. However, the manipulation tab 44 can be pressed transversely downwardly to compress the grasping member 14, disengage the teeth 46, 48, and permit the grasping member 14 to move longitudinally as shown in FIG. 6.

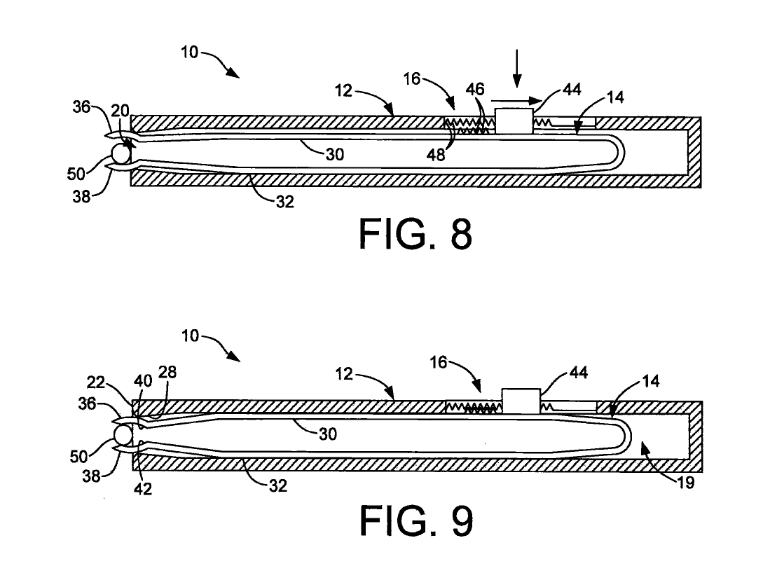

Turning now to FIGS. 6-9, the medical apparatus 10 may interact with various vascular instruments in different manners. For example, the grasping member 14 may support a wire or access sheath used, for example, during hemodialysis access structure-related medical procedures. To use the apparatus 10 in this manner, the manipulation tab 44 is pressed transversely downwardly to deflect the grasping member 14 and disengage the teeth 46, 48 of the motion control mechanism 16. While holding the manipulation tab 44 transversely downwardly, the tab 44 and the grasping member 14 are moved in the longitudinal direction as shown in FIG. 6. Eventually the grasping member 12 reaches the receiving position as shown in FIG. 7. In the receiving position, the fingers 36, 38 protrude through the first opening 20 and are spaced apart such that the vascular instrument 50 may be inserted therebetween. The manipulation tab 44 is then pressed transversely downwardly to deflect the grasping member 14 and disengage the teeth 46, 48 of the motion control mechanism 16. While holding the manipulation tab 44 transversely downwardly, the tab 44 and the grasping member 14 are moved in the longitudinal direction back toward the retracted position as shown in FIG. 8. When the fingers 36, 38 only partially protrude through the first opening 20 (that is, when the first opening 20 approximately bisects each of the fingers 36, 38), the tab 44 may be released such that the grasping member 14 enters the holding position as shown in FIG. 9. In this position, the fingers 36, 38 are disposed in the narrowest section of the internal chamber 19 defined by the tapered surface 28. As such, the fingers 36, 38 are disposed relatively close to each other, and the inner surfaces 40, 42 of the fingers 36, 38 contact the vascular instrument 50 to enclose the instrument 50 therebetween. The fingers 36, 38 may also hold the vascular instrument 50 against the longitudinal end 22 of the housing 12 in the holding position.

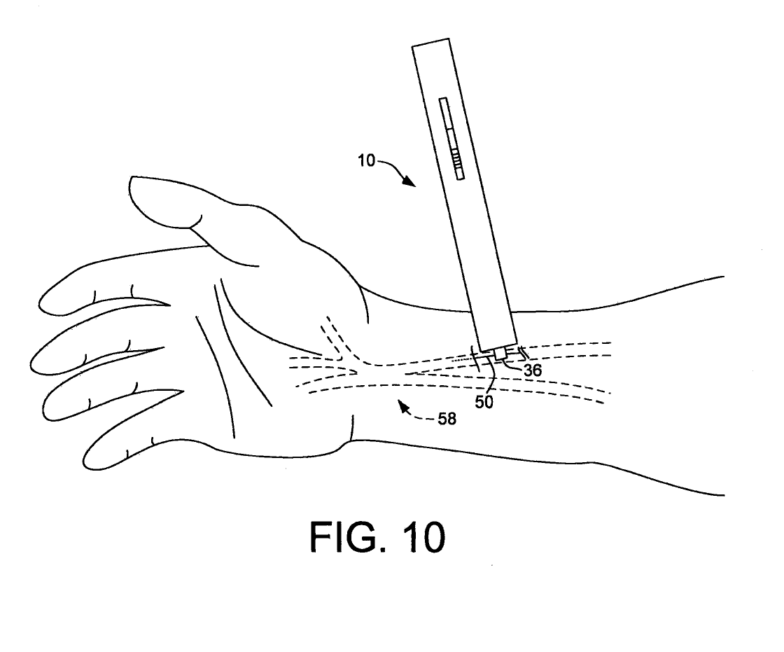

Referring to FIG. 10, after grasping the instrument 50, the practitioner may move the apparatus 10 to guide the instrument 50 toward the inflow of a hemodialysis access structure 58 to facilitate, for example, analysis or treatment of the structure 58. By grasping the end of the apparatus 10 opposite the instrument, the practitioner's hands are less likely to be exposed to radiation from simultaneous imaging procedures. Furthermore, the apparatus 10 and the instrument 50 may alternatively be used in any other type of medical procedure that involves such instruments.

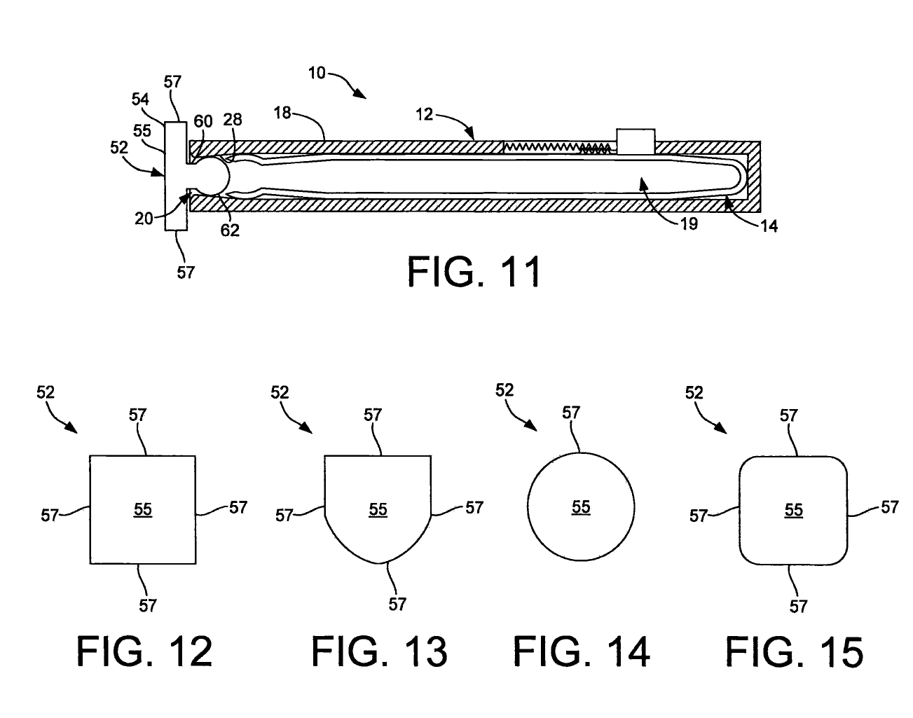

Referring to FIGS. 11-16 and as another example, the housing 12 may support a flow inhibiting instrument 52 when the grasping member 14 is disposed in the retracted position. The flow inhibiting instrument 52 includes a flow blockage portion 54 that is compressible against the vein 56 of a hemodialysis access structure 58 to prevent blood flow through the vein 56. The flow blockage portion 54 includes an end surface 55 and side surfaces 57. As viewed from the end surface 55, the flow blockage portion 54 may have various shapes, such as rectangular (FIG. 12), shield-like (FIG. 13), circular (FIG. 14), rectangular with rounded edges (FIG. 15) or the like. The flow blockage portion 54 may have a diameter or length in the range of, for example, 0.2 cm to 5 cm and a height along the side surfaces in the range of, for example, 0.1 cm to 10 cm.

The flow blockage portion 54 preferably comprises a material that is both relatively firm for effectively compressing the vein 56 and comfortable for the subject. Such materials include rubber, sponge, or the like. The flow inhibiting instrument 52 may be formed as a single piece of such a material. Alternatively, a rubber, sponge, or other cover may be fitted over the flow blockage portion 54.

The flow blockage portion 54 connects to a neck 60, and the neck 60 in turn connects to a ball portion 62 that is received in the internal chamber 19 and engages the tapered section 28 of the housing wall 18. The diameter of the ball portion 62 is slightly larger than that of the cross-sectional area of the internal chamber 19 at the tapered section 28 such that the ball portion 62 holds the flow inhibiting instrument 52 in engagement with the housing 12. To permit the ball portion 62 to initially enter the internal chamber 19 and move over the tapered section 28 of the wall 18, the ball portion 62 may engage the tapered section 28 and cause the wall 18 to deflect due to the gaps 29. Alternatively, the ball portion 62 may comprise a resilient material, such as rubber or the like.

Furthermore, the flow blockage portion 54, the neck 60, and the ball portion 62 are positioned and sized such that the flow blockage portion 54 may be positioned at different angles when connected to the housing 12.

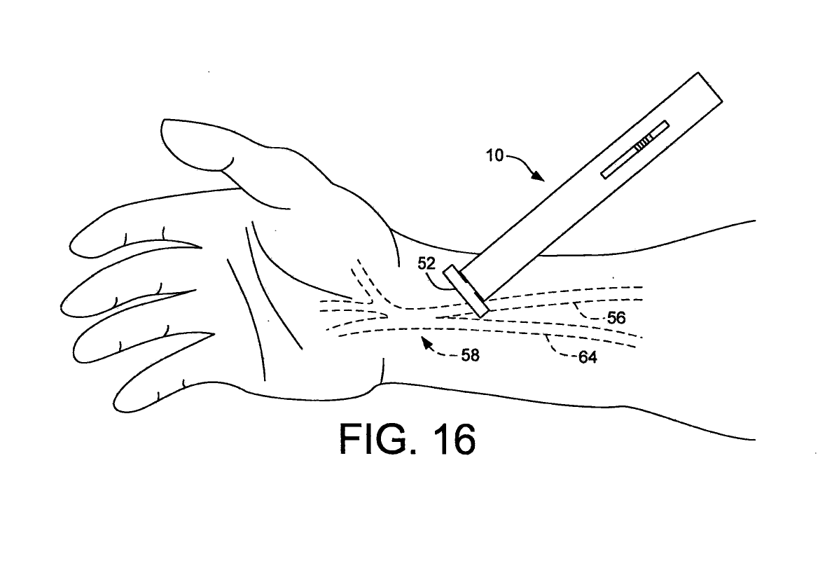

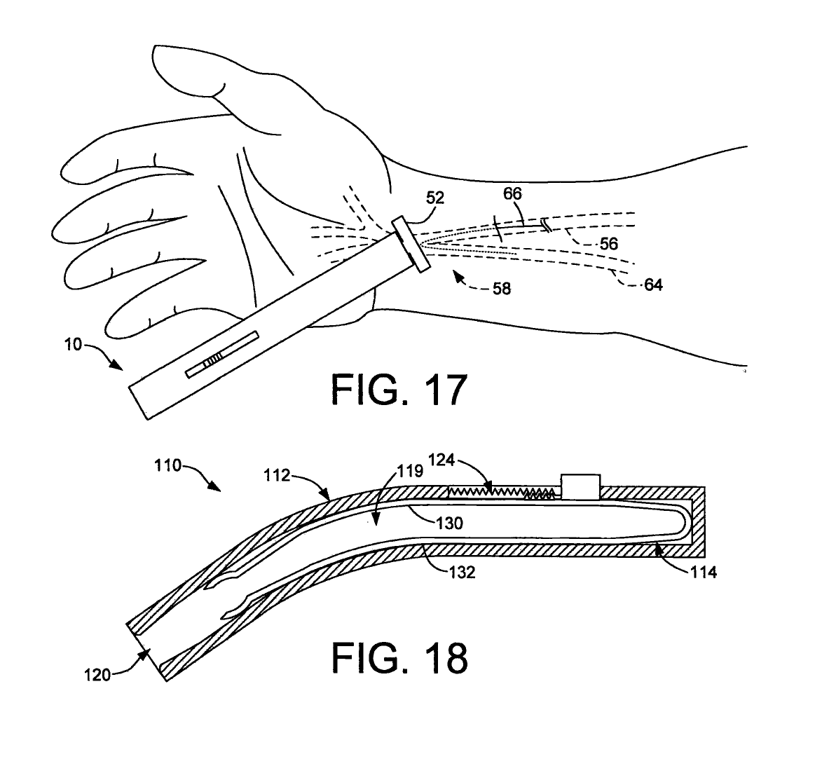

After the connection to the flow inhibiting instrument 52, the practitioner may use the apparatus 10 in several manners. For example and referring to FIG. 16, the practitioner may press the flow inhibiting instrument 52 against the subject's skin overlying the vein 56 of an arteriovenous fistula 58. Either the end surface 55 or one of the side surfaces 57 of the flow inhibiting instrument 52 may engage the subject's skin. In either case, such an action inhibits blood outflow through the vein 56, which in turn permits the practitioner to investigate blood inflow from the artery 64. As another example and referring to FIG. 17, the practitioner may press the flow inhibiting instrument 52 against the subject's skin overlying the interface distal to the anastomosis of the vein 56 and the artery 64. In this position, the flow inhibiting instrument 52 acts as a “guide” for instruments introduced to the hemodialysis access structure 58. As shown in the figures, a wire 66 may be introduced, for example, to a subject's vein 56, forced to turn at an acute angle due to the compression or the slightly altered vascular angle caused by the flow inhibiting instrument 52, and enter the artery 64.

The structure of the medical apparatus may take other forms that are not explicitly described above. For example and referring to FIG. 18, an alternative configuration of the medical apparatus 110 according to the present invention may include a curved housing 112 (that is, the longitudinal direction of the housing 112 extends along a curve between the first opening 120 and the second opening 124). In this configuration, the grasping member 114 also extends along a curve within the internal chamber 119 of the housing 112, and different portions of the arms 130 and 132 bend within the housing 112 as the grasping member 114 moves from the retracted position to the receiving and holding positions. Other features of the medical apparatus 110 are generally similar to those described above.

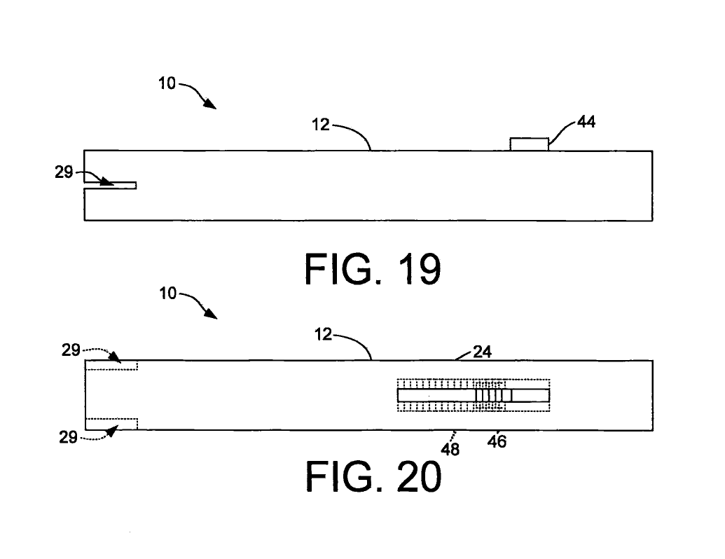

As another example and referring now to FIGS. 19 and 20, in another alternative embodiment the housing wall 18 may include one or more gaps 29 proximate the first opening 20 and extending in the longitudinal direction. These gaps 29, which may be disposed at different sides or locations from the first opening 20 and/or at different angles on the housing wall 18, permit the wall 18 to deflect to a limited extent and increase the size of the first opening 20, which in turn permits the ball portion 62 of the flow inhibiting instrument 52 to enter the housing 12 if the ball portion 62 does not comprise a resilient material.

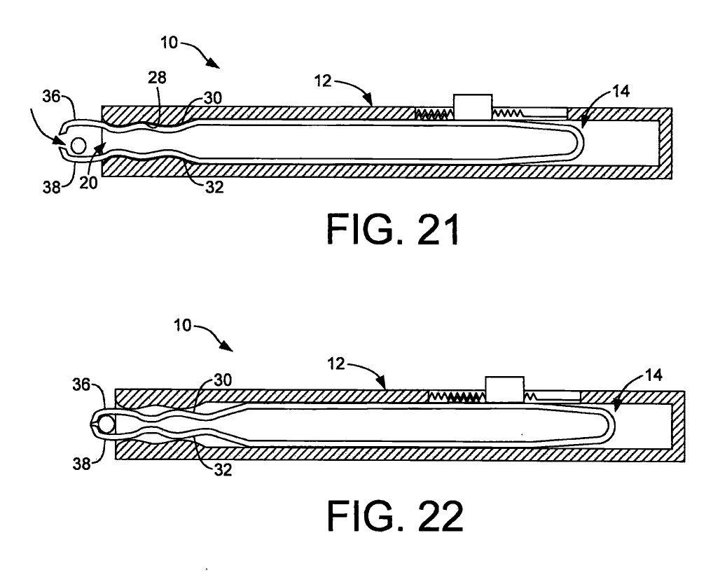

As yet another example, the housing 12 and the grasping member 14 may have other shapes. As shown in FIGS. 21 and 22, the tapered section 28 of the internal chamber 19 may have a curved shape that, proceeding from the opening 20, first tapers inwardly, then outwardly, and then inwardly and outwardly again. The relatively wide portion of the tapered section 28 (that is, near the middle where it tapers outwardly and then inwardly) is appropriate for receiving the ball portion 62 of the flow inhibiting instrument 52. Furthermore, the arms 30, 32 of the grasping member 14 may have a matching shape that interfits with the tapered section 28 when the grasping member 14 is in the receiving position (FIG. 21). Even further still, the fingers 36, 38 may be shaped and sized to contact each other in the holding position (FIG. 22).

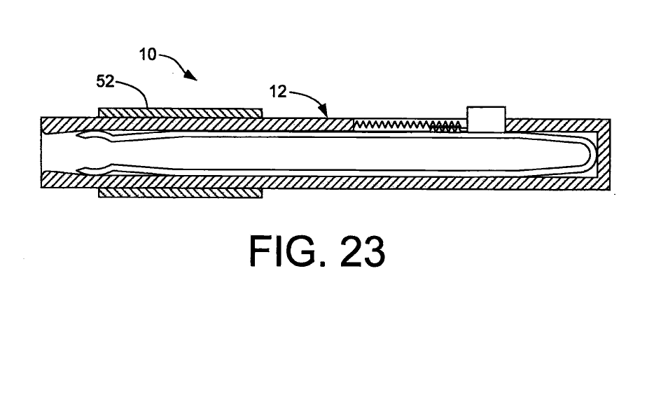

As yet another example and referring to FIG. 23, the flow inhibiting instrument 52 may have a sleeve-like hollow shape that fits over the housing 12. Such a flow inhibiting instrument 52 may be pressed against a subject's skin to inhibit blood flow or guide an instrument as described above.

In other alternative configurations, the motion control mechanism 16 may take other forms. For example, the upwardly facing teeth 46 and the downwardly facing teeth 48 could be other types of motion control members. In some cases, a first motion control member is a pin supported by the grasping member 14 and the second motion control member is a plurality of pin-receiving slots defined by the housing 12. As another example, the upper arm of the grasping member could include a high friction pad that engages the wall 18 to inhibit translation of the grasping member 14 relative to the housing 12. As yet another alternative embodiment, the motion control mechanism 16 could include a pair of magnets or a single magnet and a magnetically attracted material (for example, metal).

It should be apparent that the present invention provides a medical apparatus that facilitates effective blockage of venous outflow from a hemodialysis access structure. In addition, the medical apparatus is capable of firmly grasping various medical instruments. By holding the end of the apparatus opposite the instrument, a practitioner may position his or her hands outside of a medical imaging field and thereby reduce radiation exposure to the hands.

The various configurations presented above are merely examples and are in no way meant to limit the scope of this disclosure. Variations of the configurations described herein will be apparent to persons of ordinary skill in the art, such variations being within the intended scope of the present application. In particular, features from one or more of the above-described configurations may be selected to create alternative configurations comprised of a sub-combination of features that may not be explicitly described above. In addition, features from one or more of the above-described configurations may be selected and combined to create alternative configurations comprised of a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

What is claimed is:

1. A medical apparatus for supporting a vascular instrument configured to interact with a portion of a cardiovascular system of a subject, the apparatus comprising:
a housing defining an internal chamber, the internal chamber having an opening defined at an end of the housing;
a grasping member supported by the housing within the internal chamber, the grasping member being translatable relative to the housing to at least partially protrude from the internal chamber through the opening and support the vascular instrument; and
a motion control mechanism supported by the housing and being engagable to inhibit translation of the grasping member relative to the housing.

2. The medical apparatus of claim 1, wherein the opening is configured to detachably receive a flow inhibiting instrument configured to engage the portion of the vascular system and thereby inhibit blood flow therethrough.

3. The medical apparatus of claim 1, wherein the grasping member is translatable from a receiving position to a holding position, in the receiving position the grasping member at least partially protrudes from the internal chamber through the opening and supports the vascular instrument, and in the holding position the grasping member fixedly supports the vascular instrument.

4. The medical apparatus of claim 3, wherein the grasping member is translatable to and from a retracted position in which grasping member is completely disposed within the internal chamber, and wherein the holding position is located between the receiving position and the retracted position.

5. The medical apparatus of claim 1, wherein the motion control mechanism is engagable in a receiving position in which the grasping member at least partially protrudes from the internal chamber and supports the vascular instrument.

6. The medical apparatus of claim 1, wherein the motion control mechanism is engagable in a holding position in which the grasping member fixedly supports the vascular instrument.

7. The medical apparatus of claim 1, wherein the opening faces in a longitudinal direction of the housing and the grasping member is translatable in the longitudinal direction, and the motion control mechanism is engagable in a transverse direction substantially parallel to the longitudinal direction.

8. A medical apparatus for supporting a first vascular instrument configured to interact with a portion of a cardiovascular system of a subject and for supporting a second vascular instrument configured to inhibit blood flow through the portion of the cardiovascular system, the apparatus comprising:
a housing defining an internal chamber, the internal chamber having an opening defined at an end of the housing and configured to detachably receive the second vascular instrument;
a grasping member supported by the housing within the internal chamber and being movable from a retracted position to a receiving position relative to the housing;
a motion control mechanism supported by the housing and being engagable to inhibit translation of the grasping member relative to the housing; and
wherein in the receiving position at least a portion of the grasping member protrudes from the internal chamber through the opening and is configured to support the first vascular instrument.

9. The medical apparatus of claim 8, wherein the grasping member includes at least two fingers disposed on opposite sides of the grasping member and facing one another, and in the receiving position the fingers protrude from the opening and are configured to support the first vascular instrument.

10. The medical apparatus of claim 9, wherein the grasping member is movable to and from a holding position in which the grasping member fixedly supports the first vascular instrument.

11. The medical apparatus of claim 10, wherein the fingers are spaced apart by a first distance in the receiving position and a second distance in the holding position, and the second distance is less than the first distance.

12. The medical apparatus of claim 10, wherein the housing tapers inwardly proximate the opening to urge the fingers toward each other when the grasping member moves toward the holding position.

13. The medical apparatus of claim 8, wherein in the retracted position, the grasping member is disposed completely within the internal chamber, and the opening is only configured to receive the second vascular instrument when the grasping member is disposed in the retracted position.

14. The medical apparatus of claim 8, wherein the grasping member includes a manually engagable manipulation tab being movable relative to the housing to move the grasping member from the retracted position to the receiving position.

15. A medical apparatus configured to interact with a portion of a cardiovascular system of a subject, the apparatus comprising:
an elongated housing; and
a flow inhibiting instrument detachably supported by the housing and being configured to engage the portion of the cardiovascular system and inhibit blood flow therethrough.

16. The medical apparatus of claim 15, wherein the housing defines an internal chamber, the internal chamber having an opening detachably receiving the flow inhibiting instrument defined at an end of the housing, further comprising a grasping member movably supported by the housing within the internal chamber; and wherein when the flow inhibiting instrument is detached from the housing the grasping member is movable such that at least a portion of the grasping member protrudes from the internal chamber through the opening and is configured to support an elongated vascular instrument.

17. The medical apparatus of claim 16, wherein the grasping member includes a flexible body that deflects as the grasping member protrudes through the opening.

18. The medical apparatus of claim 16, further comprising:
a first motion control member supported by the housing; and
a second motion control member supported by the grasping member and being engagable with the first motion control member to inhibit movement of the grasping member relative to the housing.

19. The medical apparatus of claim 18, wherein the second motion control member normally engages the first motion control member.

20. The medical apparatus of claim 16, wherein the grasping member is translatable from a receiving position to a holding position, in the receiving position the at least a portion of the grasping member protrudes from the opening and is configured to support the elongated vascular instrument, and in the holding position the grasping member is configured to fixedly support the elongated vascular instrument.

21. The medical apparatus of claim 20, wherein the grasping member includes a pair of opposed fingers configured to engage the elongated vascular instrument therebetween, and wherein the fingers are movable toward each other to engage the elongated vascular instrument when the grasping member is in the receiving position.

22. The medical apparatus of claim 21, wherein the fingers contact each other in the holding position.
